(19) United States
(12) Patent Application Publication
(10) Pub. No.: US 2019/0255321 A1
(43) Pub. Date: Aug. 22, 2019

(54) SKIN TREATMENT DEVICE COMPRISING A PULSE MODULATOR

(71) Applicant: L'OREAL, Paris (FR)

(72) Inventor: Thi Hong Lien PLANARD-LUONG, Chevilly La Rue (FR)

(73) Assignee: L'OREAL, Paris (FR)

(21) Appl. No.: 16/333,786

(22) PCT Filed: Sep. 19, 2017
(86) PCT No.: PCT/EP2017/073678

§ 371 Date: Mar. 15, 2019

(30) Foreign Application Priority Data
Sep. 20, 2016 (FR) 1658824

(51) Int. Cl. A61N 1/32; A61M 37/00; A61F 7/00; A61N 5/06; A61N 1/04; H03K 7/02; H03K 7/08

(52) U.S. Cl. CPC A61N 1/328; A61N 1/327; A61M 37/0092; A61F 7/00; A61N 5/0616; A61N 5/062; A61M 2205/3368; H03K 7/02; H03K 7/08; A61F 2007/0052; A61M 2037/0007; A61M 2205/3317; A61N 1/0448

(57) ABSTRACT

The invention relates to a device for the cosmetic treatment of keratin materials with the aid of an electric current, comprising at least one generator (1) which produces an elementary signal formed by a high-frequency signal which is amplitude-modulated by a medium-frequency signal and at least one electrode (5) connected to an output of the generator (1) and which can be moved over a body area to be treated and a counterelectrode (6), the generator (1) comprising an elementary signal processing unit comprising a pulse modulator (4) which delivers a low pulse-frequency current wave.

The present invention relates to devices for performing a cosmetic treatment of keratin materials.

More generally, a cosmetic product is a product as defined in Regulation (EC) No 1223/2009 of the European Parliament and Council of 30 Nov. 2009 relating to cosmetic products.

The invention relates in particular to devices for treating keratin materials with the aid of an electric current.

The expression “human keratin materials” is understood as meaning mainly the skin, notably of the body or of the face, or even the scalp, the nails or the hair.

### TECHNOLOGICAL BACKGROUND

Various types of cosmetic treatment with an electric current exist.

It is known that the application of an electric current to the skin can promote the penetration of an active agent. It is thus known to treat human keratin materials with the aid of iontophoretic devices. Iontophoresis allows the diffusion of active agents through the skin by virtue of electrical stimulation in a non-invasive manner. The current administered can be adjustable in terms of intensity and polarity (anodic or cathodic current). The transcutaneous diffusion of the molecules via iontophoresis is based on two principles, namely electrorepulsion and electroosmosis.

The radiofrequency is based on the emission of very high frequency electromagnetic waves passing through the skin, which produce heat in the subcutaneous tissues. The heat produced acts deep down, without burning the surface, i.e. the skin. It is possible to heat up to 55° C. deep down, which stimulates the fibroblasts, which manufacture new elastin and collagen fibres. In parallel, the heat induces shrinkage of the pre-existing collagen fibres.

This revolutionary technique treats skin deep down, and stimulates the production of new collagen, which improves the firmness and tautness of the skin.

Electroporation (also known as needle-free mesotherapy) is a method which induces opening of the skin's water channels (electropores) via the effect of specific waveforms. Better transport of the active principles of the cosmetics used is obtained. Various techniques exist, depending on the waveform and the application methods used. Electroporation has a shorter duration of application than conventional iontophoresis, it is entirely painless and allows controlled transport of large amounts of active principles.

The use of electrostimulation in aesthetics makes it possible to firm and tonify certain slackened muscles of the face and body. The particular feature of nerve and muscle cells is that they are excitable. This property allows them to respond to an electrical “stimulus”: when this stimulus is initiated on a nerve cell, it propagates very quickly along the membrane thereof. After having crossed the “neuromuscular junction”, it travels across the membrane of the muscle cells, and the excitation is then transmitted deep down and triggers contraction.

Several types of appliances for treating the skin with microcurrents which supply electrical pulses of different forms and frequencies have already been proposed.

U.S. Pat. No. 8,731,657 describes a device comprising a microcontroller configured to generate a plurality of high and low frequency electromagnetic waveforms of different amplitudes.

U.S. Pat. No. 6,684,107 describes a wireless device comprising a generator for periodically generating a microcurrent with a frequency of less than 16 Hz.

There is a need to further improve devices for cosmetic treatment with the aid of an electric current which promote the penetration of active agents so as to increase the efficacy of the various known technologies.

There is a need to benefit from a device for treating keratin materials with the aid of an electric current, which exhibits good efficacy and can be used with comfort and in total safety, including its use for dynamic treatment.

There is also a need to benefit from such a safe device, which limits the tingling, burning or heating of the treated zone.

There is still a need for such a device which improves the quality, firmness, complexion and texture of the skin and which reduces the wrinkles and sheen.

There is still a need for such a device for which the wear of the electronic components is reduced.

### Definition of the Invention

According to one of its aspects, one subject of the invention is thus a device for the cosmetic treatment of keratin materials with the aid of an electric current, comprising at least one generator which produces an elementary signal formed by a high-frequency signal which is amplitude-modulated by a medium-frequency signal and at least one electrode connected to an output of the generator and which can be moved over a body area to be treated and a counterelectrode, the generator comprising an elementary signal processing unit comprising a pulse modulator which delivers a low pulse-frequency current wave.

By means of the pulse modulator, the signal obtained is attenuated relative to the elementary signal. It is markedly less aggressive to the user, thus limiting the risks of tingling, heating or burning of the treated body area.

The device according to the invention is simple to produce since the pulse modulator can even be incorporated into the current generator.

The intensity of the signal can also be readily controlled and modulated, for greater efficiency of the treatment.

In particular, the process according to the invention is an electrostimulatory process.

The invention also relates to a cosmetic process comprising the use of a device described previously.

Power Supply System

The device comprises a power supply system for exposing the keratin materials to an electrical treatment current in a zone of application of the composition.

A “power supply system” is understood to be an electrical assembly that is able to induce a potential difference between one or more electrodes and at least one counterelectrode. If the application end piece is placed on the face and if the counterelectrode is held in one hand, the potential difference is established between the face and the hand.

Combination of the High-Frequency Signal and of the Medium-Frequency Signal

To combine the two signals of different frequencies, a DC-CDI circuit or a chopper run at high frequency and receiving the medium-frequency signal as input may be envisaged, for example.

Pulse Width Modulator PWM

The main advantage of a PWM modulator is that it limits the heating of electronic components. In PWM, the power supplied to the end piece is either maximal, or zero. When it is maximal, there is no need to dissipate residual power. When it is zero, there is no need either to dissipate power, since none is supplied at all. Another advantage of the PWM is that the voltage applied to the end piece for a time 6t is Vcc. This is sufficient to bring about an electrical treatment of the skin.

(i) Frequency

The PWM power actuating command is highly associated with the notion of frequency. In order for the impression of a constant mean current value to appear, the on/off alternance of the signal needs to be fast enough for it not to be noticed by the user.

To aid the comprehension, FIGS. 1 to 3 represent three simple theoretical examples of rectangular analogue signals U(t). The maximum voltage is noted as Vₐ and the calculated mean voltage is noted as Vₘₑₐₙ. For the three signals, the duty cycle was calculated as indicated below.

(ii) Duty Cycle

The duty cycle refers to the ratio:

α=100×tₕ/T

expressed as a percentage and measured on the curve U(t).
tₕ is the duration of a pulse.
T is the period of the signal.
In FIG. 1, the duty cycle is close to 0%.
In FIG. 2, the duty cycle is close to 50%.
In FIG. 3, the duty cycle is close to 100%.
FIG. 4 specifies on a simple theoretical signal example the determination of tₕ and T.

(iii) Production of the PWM Signal

The most simple means for generating a PWM signal is the intersective method, which requires only a sawtooth or triangular waveform, generated, for example, by means of an oscillator and a comparator.

Many digital circuits can generate PWM signals (for example, many microcontrollers have PWM outputs). They generally use a counter which periodically increments (it is connected directly or indirectly to the circuit clock) and is reset at the end of each PWM period. When the value of the counter is greater than a reference value, the PWM output changes state.

Pulse Amplitude Modulator

The simplest form of analogue pulse modulation is pulse amplitude modulation (PAM). As its name indicates, it consists in modulating the amplitude of a train of equidistant pulses, as a function of the values of samples of an analogue signal. It is not at all a question of modulating the parameters of a carrier, but indeed of affecting the starting signal. FIG. 5 illustrates the definition of a pulse amplitude modulator. It shows an example of PAM modulation by rectangular pulses. The PAM signal m(t) is constructed while keeping the signal at a constant value.

m_{τ′}(t) is the modulation of the signal as a function of time over a pulse duration τ′.

m_{τ}(t) is the modulation of the signal as a function of time over a pulse duration τ.

T is the period of the pulsed signal.

A circuit which limits itself to the use of signals for the purpose of an effective treatment comprises an analogue-digital conversion, followed, if need be, by a digital filter intended to partially adapt the frequency properties to the subsequent treatment. The conversion chain is preceded by a low-pass analogue filter which adjusts the passband of the signal as a function of the characteristic sampling frequency.

Application End Piece

The application end piece may or may not be conductive.

It may comprise several application members, notably in the form of balls or spikes. The multiplicity of application members promotes good distribution of the composition in a continuous manner, and improves the ionization of the composition during the treatment, where appropriate. The multiplicity of application members also makes it possible to improve the massaging effect during treatment.

Advantageously, the application end piece comprises at least one ball that is movable about a centre of rotation.

As a variant, the application members may have any profile, notably a cylindrical profile, for example in the form of rollers, or a non-cylindrical profile, for example an ovoid or discoid shape.

The application members may be made of plastic or metal.

The application end piece may be a pad.

The outer surface of the end piece may be totally inert from a chemical point of view with regard to the composition applied and the keratin materials. The outer surface may be covered with a varnish. The outer surface may be polished. The outer surface may comprise a biocidal material, where appropriate.

Electrodes

The electrode may be positively charged (anode) or negatively charged (cathode). This electrode may be located in the application end piece, so as to ensure the passage of the electric current to the skin. If the electrode is located inside the end piece, it does not come directly into contact with the keratin materials, and is referred to as a remote electrode.

An electrode may be in the form of a ball, spike, stud, tongue, for example. The device may comprise a single or several electrodes.

The electrode may be flat, for example in the form of a flat disc or of a polygon.

The electrode may be hollow, being formed for example by stamping or bending an electrically conductive metal sheet. The electrode may be porous. The electrode may be formed by a surface treatment for making the first zone conductive.

A “counterelectrode” is understood to be an electrode that is charged with an opposite sign to the electrode: negative, positive or alternating or connected to the earth of the generator. In general, said counterelectrode is located on the body of the device or on a handpiece. The counterelectrode is intended to come into contact with an area of the body of the person undergoing the care. In one embodiment, the counterelectrode is located on the end piece. If this is the case, it is separated from the electrode by an insulating space.

If the electrode is housed inside the end piece, it may notably be spaced apart from the outer wall of the application end piece by a distance of between 0.2 mm and 5 mm. This distance is the shortest measurable distance between the electrode and the outer surface of the application end piece. It is measured between any points of the electrode and of the outer surface of the application end piece, as long as the distance measured is the shortest distance.

Electrical Parameters

The electrical power source may comprise any cell or any accumulator or any transformer. The voltage between the terminals is, for example, between 1.2 V and 24 V, preferably between 1.2 and 3.3 V. Where appropriate, the passage of the current can create spot heating.

The peak voltage of the pulses may range up to 400 V, but the energy delivered does not exceed 80 mJ per pulse or the power of the pulses does not exceed 5 W.

Complementary Cosmetic Treatments

Advantageously, the device comprises a source of heat, light, for setting the application end piece in motion, notably a sonic, ultrasonic or iontophoretic vibration.

Composition

The device according to the invention may comprise a reservoir containing a cosmetic composition. The reservoir may or may not be removable.

Preferably, the cosmetic composition has a viscosity, measured at 25° C. and at a normal atmospheric pressure of 1.013×10⁵ Pa, of less than 0.5 Pa·s, more preferably less than 0.3 Pa·s, better still less than 0.2 Pa·s, notably in the range from 0.1 to 0.2 Pa·s, better still from 0.04 to 0.175 Pa·s.

The viscosity of the composition is measured at 25° C. and at a normal atmospheric pressure of 1.013×10⁵ Pa using a Rheomat 180 (from the company Lamy), equipped with an MS-R1, MS-R2, MS-R3, MS-R4 or MS-R5 spindle chosen depending on the consistency of the composition, and rotating at a rotation speed of 200 rpm. The measurement is taken after 10 minutes of rotation. The viscosity measurements are taken at most 1 week after production.

In the context of the invention, the spindles RS-R3 or MS-R2 or, for the most fluid compositions, MS-R1 will be used.

It is possible to use a cosmetic or dermatological composition with the device. The application may take place before or after the treatment.

The composition(s) used may be in any form, for example in the form of an aqueous solution, an oil, an emulsion, a powder or a gel. The composition(s) used may also be sprayed onto the skin.

When the composition(s) used are in the form of a powder or gel, the latter can take on the shape of the electrode to which it is applied, as mentioned above. One or more compositions can be applied. To this end, the reservoir may comprise several compartments.

The composition(s) may comprise an active agent.

Advantageously, the composition is chosen from:
a face care or body care composition, comprising in particular an active agent chosen from humectant or moisturizing active agents, anti-ageing active agents, for example, depigmenting active agents, active agents that act on cutaneous microcirculation or seboregulating, anti-acne, filling, vitamin active agents;
a temporary, semi-permanent or permanent composition for making up the face or body,
a hair composition, in particular a composition for washing the hair, for hair care or conditioning, for temporary form retention or shaping of the hair, for the temporary, semi-permanent or permanent dyeing of the hair, or for relaxing or permanent-waving, in particular a composition for relaxing, dyeing or bleaching the roots and hair,
a composition for the scalp, in particular an antidandruff composition, a composition for preventing hair loss or for promoting regrowth of the hair, an anti-seborrhoeic, anti-inflammatory, anti-irritation or soothing composition, a mark-preventing composition or a composition for stimulating or protecting the scalp.

The device may be used in various cosmetic or dermatological treatments, for example for combating wrinkles, herpes, acne or for redensifying the skin or the hair.

### PREFERRED EMBODIMENTS

Preferably, the device according to the invention has one or more of the following features, on their own or in combination:
the pulse modulator has a switching frequency of between 1 Hz and 100 Hz. This frequency is adapted to produce a particularly efficient signal.
the pulse modulator is a PWM modulator delivering a signal U(t) defining a duty cycle of between 1% and 75%. With this duty cycle, the signal intensity is satisfactory.
the pulse modulator delivers a low-frequency modulation having a sinusoidal, triangular, square or rectangular form. These forms are ideal for obtaining a suitable elementary signal.
the pulse modulator delivers a signal U(t) having a pulse duration of between 4 ms and 8 ms. Thus, the user does not perceive the momentary absence of current on his skin.
a microcontroller generates the pulse modulator and a voltage regulator capable of adjusting a maximum voltage of the current wave as a function of an impedance measured between the electrode and the body area. The circuit is easy to produce and compact.
the high-frequency signal has a frequency of between 1 MHz and 100 MHz, preferably between 1 MHz and 10 MHz and more preferentially between 3 MHz and 5 MHz.
the medium-frequency signal has a frequency of between 1 kHz and 1000 kHz, preferably between 10 kHz and 700 kHz and more preferentially between 300 kHz and 500 kHz.
the generator comprises a DC-CDI (capacitive discharge ignition) circuit run at high frequency and receiving as input a medium-frequency voltage. The device is produced with components that are available on the market and readily accessible.
the device comprises a source of heat, light, for setting the application end piece in motion, notably a sonic, ultrasonic or electroporation vibration. The treatment may be improved and, what is more, more efficient.
the device comprises a resistive or capacitive sensor for controlling a movement, a temperature, an impedance or a pH. The safety of the device is increased.

### DESCRIPTION OF THE FIGURES

The invention will be better understood on reading the following detailed description of non-limiting illustrative embodiments of the invention and on examining the appended, schematic and partial drawing, in which:

The invention is not limited to the examples illustrated. In particular, the features of the various exemplary embodiments which have just been described can be combined with each other.

FIG. 6 shows an example of an elementary signal U(t) in damped sinusoidal form.

FIGS. 7 and 8 show examples of pulse frequency waves.

FIG. 9 shows an exemplary embodiment of a circuit according to the invention.

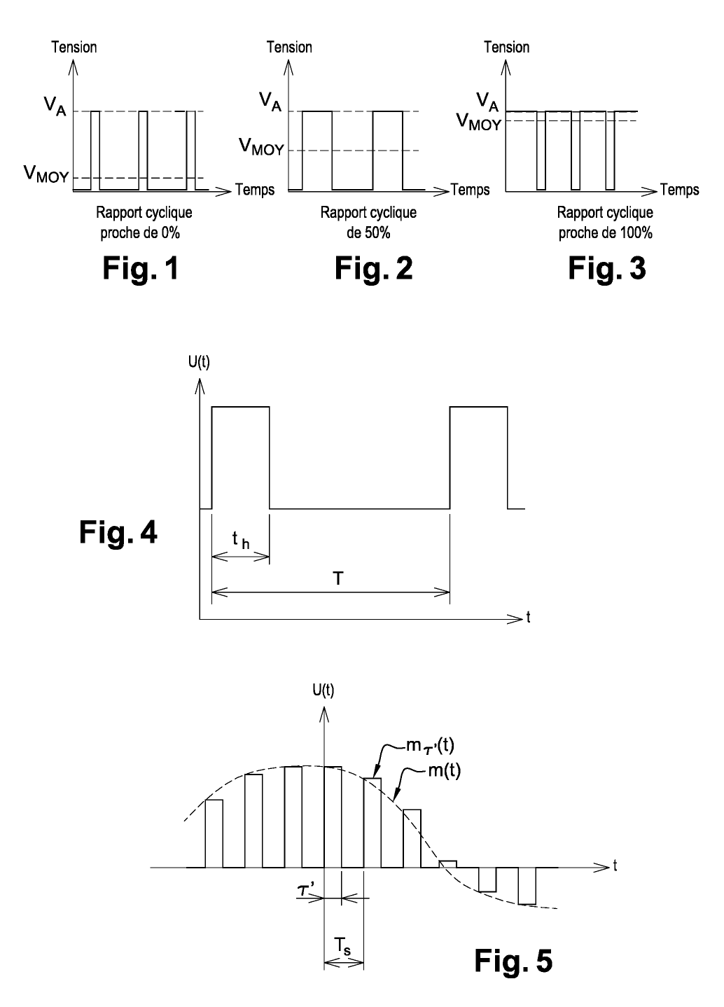

FIG. 6 shows a symmetrical damped sinusoidal signal of high frequency equal, for example, to 3 MHz.

It is amplitude-modulated by a medium-frequency signal, for example of 358 kHz, 620 kHz, 720 kHz.

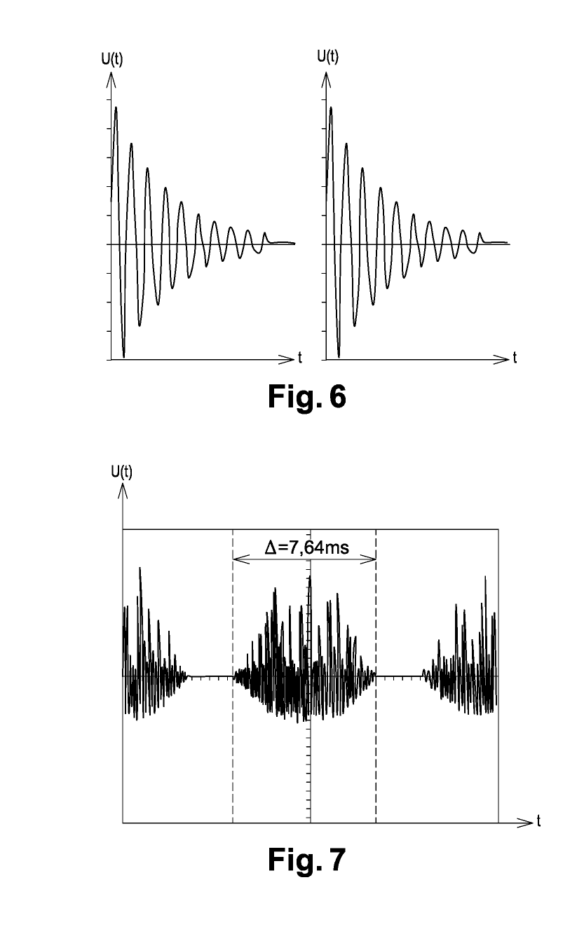

FIG. 7 is a 100 Hz sinusoidal current wave with a peak voltage of 630 V obtained as the output from a pulse modulator.

FIG. 8 is a 100 Hz sinusoidal current wave with a peak voltage from 50 V to 340 V obtained as the output from a pulse modulator.

The signals generated by a skin treatment appliance give improved treatment efficiency and less tingling and heating on the treated area.

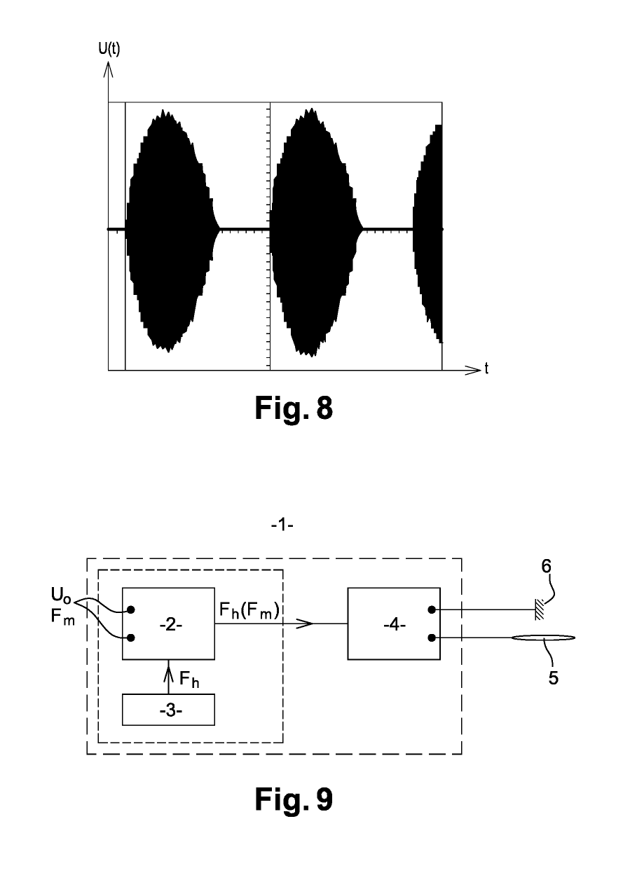

FIG. 9 shows a generator 1 comprising a DC-CDI circuit 2 receiving as input an alternating voltage U₀ at a frequency of medium frequency Fₘ, for example 1000 kHz. This alternating voltage may be taken from the current distribution network, for example via a step-down transformer located outside the generator 1. Moreover, an oscillator 3 of high frequency Fₕ, for example 3 MHz, may be connected to the control input of the DC-CDI circuit 2. The result of this is that the output signal from the DC-CDI circuit 2 is a high-frequency signal Fₕ (3 MHz) which is amplitude-modulated by the medium-frequency signal Fₘ(1000 kHz) known as the elementary signal. The high-frequency oscillations, of 3 MHz in this instance, change in amplitude inside a medium-frequency envelope (1000 kHz), for example like the signal shown in FIG. 6.

The elementary signal is applied to a pulse modulator 4, one output terminal of which is connected to an electrode 5, for example a metal electrode, whereas the other output terminal is connected to a counterelectrode 6 intended to come into contact with the person and serving as earth. The electrode 5, for its part, will be moved slowly over the area of the epidermis to be treated. The signal available at the output of the pulse modulator 4 is a low pulse-frequency current wave, for example like that shown in FIG. 7 or 8.

1: Device for the cosmetic treatment of keratin materials with the aid of an electric current, comprising at least one generator which produces an elementary signal formed by a high-frequency signal which is amplitude-modulated by a medium-frequency signal and at least one electrode connected to an output of the generator and which can be moved over a body area to be treated and a counterelectrode,
wherein the generator comprises an elementary signal processing unit comprising a pulse modulator which delivers a low pulse-frequency current wave.

2: The device according to claim 1, wherein the pulse modulator is a pulse width modulator PWM or a pulse amplitude modulator PAM.

3: The device according to claim 1, wherein the pulse modulator has a switching frequency of between 1 Hz and 100 Hz.

4: The device according to claim 1, wherein the pulse modulator is a PWM modulator delivering a signal U(t) defining a duty cycle of between 1% and 75%.

5: The device according to claim 1, wherein the pulse modulator delivers a low-frequency modulation having a sinusoidal, triangular, square or rectangular form.

6: The device according to claim 1, wherein the pulse modulator delivers a signal U(t) having a pulse duration of between 4 ms and 8 ms.

7: The device according to claim 1, wherein a microcontroller generates the pulse modulator and a voltage regulator capable of adjusting a maximum voltage of the current wave as a function of an impedance measured between the electrode and the body area.

8: The device according to claim 1, wherein the high-frequency signal has a frequency of between 1 MHz and 100 MHz, preferably between 1 MHz and 10 MHz and more preferentially between 3 MHz and 5 MHz.

9: The device according to claim 1, wherein the medium-frequency signal has a frequency of between 1 kHz and 1000 kHz, preferably between 10 kHz and 700 kHz and more preferentially between 300 kHz and 500 kHz.

10: The device according to claim 1, wherein the generator comprises a DC-CDI (capacitive discharge ignition) circuit run at high frequency and receiving as input a medium-frequency voltage.

11: The device according to claim 1, wherein it comprises a source of heat, light, for setting the application end piece in motion, notably a sonic, ultrasonic or iontophoretic vibration.

12: The device according to claim 1, wherein it comprises a resistive or capacitive sensor for controlling a movement, a temperature, an impedance or a pH.

13: The device according to claim 1, wherein it comprises a cosmetic product reservoir.

14: The device according to claim 1, wherein it is an electrostimulatory device.

15: Cosmetic process, wherein it comprises the use of a device according to claim 1.
